(19) United States
(12) Patent Application Publication
(10) Pub. No.: US 2009/0138036 A1
(43) Pub. Date: May 28, 2009

(54) BIOABSORBABLE DETACHABLE COIL AND METHODS OF USE AND MANUFACTURE

(75) Inventors: Christopher Nardone, Chelmsford, MA (US); Marcia Buiser, Marlborough, MA (US)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: Boston Scientific Scimed, Inc.

(21) Appl. No.: 12/255,494

(22) Filed: Oct. 21, 2008

Related U.S. Application Data
(60) Provisional application No. 60/981,615, filed on Oct. 22, 2007.

(51) Int. Cl. A61M 29/00

(52) U.S. Cl. 606/200

(57) ABSTRACT

Described herein are various implant delivery methods and systems that include an implantable medical device secured to a pusher wire with biomaterial. The biomaterial is selected to controllably decouple the implantable medical device from the pusher wire after exposure to a flushing agent.

### CROSS-REFERENCE TO RELATED APPLICATION

This application claims benefit to U.S. Provisional Application No. 60/981,615, filed Oct. 22, 2007, the contents of which are incorporated by reference in its entirety.

### BACKGROUND

The endovascular treatment of a variety of maladies throughout the body is an increasingly important form of therapy. One such procedure uses embolizing coils to occlude a target site by posing a physical barrier to blood flow and/or by promoting thrombus formation at the site. Such treatments can be useful where it is desired to reduce vascularization, including treatments for aneurisms and cancer.

Coils have typically been placed at a desired site within the vasculature using a catheter and a pusher. As a first step, a flexible, small diameter catheter can be guided to the target site through the use of guidewires or by flow-directed means such as balloons placed at the distal end of the catheter. Once the target site has been reached, the catheter lumen is cleared by removing the guidewire (if a guidewire has been used), and the coil is placed into the proximal open end of the catheter and advanced through the catheter with a pusher. Pushers are essentially specialized wires having a distal end that is adapted to engage and push the coil through the catheter lumen as the pusher is advanced through the catheter. When the coil reaches the distal end of the catheter, it is discharged from the catheter by the pusher into the vascular site.

Several techniques have been developed to enable more accurate placement of coils within a vessel. In one technique, the coil is bonded via a metal-to-metal joint to the distal end of the pusher. The pusher and coil are made of dissimilar metals. The coil-carrying pusher is advanced through the catheter to the site, and a small electrical current is passed through the pusher-coil assembly. The current causes the joint between the pusher and the coil to be severed via electrolysis. The pusher can then be retracted leaving the detached coil at an exact position within the vessel. In addition to enabling accurate coil placement, the electric current can facilitate thrombus formation at the coil site. A perceived disadvantage of this method is that the electrolytic release of the coil requires a period of time for the metal-to-metal joint to dissolve, so that more rapid detachment of the coil from the pusher cannot occur.

Another technique for detaching an embolic coil uses a mechanical connection between the coil and the pusher. For example, one such device uses interlocking clasps that are secured to each other by a control wire that extends the length of the catheter. Retraction of the control wire uncouples the coil from the pusher. While mechanical connections between coils and pusher wires provide for quick detachment, such detachable coils require additional control mechanisms (e.g., control wires) to deploy the coil.

Accordingly, while conventional systems provide effective coil delivery, self-detaching coils allowing uncomplicated coil delivery and reducing the chance of premature deployment or jamming would be beneficial.

### SUMMARY

In accordance with the invention, disclosed herein are implantable device delivery systems, methods of use, and processes for their manufacture. In one embodiment, a system includes an implantable device mated to a pusher wire with biomaterial, wherein the biomaterial is configured to inhibit unwanted or premature detachment of the implantable device during delivery. In one aspect, the presence of a flushing agent allows decoupling of the implantable device from the pusher wire.

A variety of flushing agents can be used with the systems described herein. In one aspect, the flushing agent is delivered to the biomaterial when detachment of the implantable device is desired. Alternatively, or additionally, the flushing agent is an in vivo substance, such as, for example, blood.

The biomaterial can be applied to various locations on the pusher wire and implantable device. In an illustrated embodiment, the biomaterial coats a portion of the outer surfaces of the implantable device and the pusher wire. The coating can comprise one or more layers of biomaterial. Additionally, each layer can comprise the same or different biomaterial.

The biomaterial can also secure the pusher wire and the implantable device at their respective ends. For example, the distal end of the pusher wire and the proximal end of the implantable device can be secured with a mass of biomaterial.

Where the biomaterial is positioned on the outer surface of the pusher wire and/or implantable device, the outer surface of the biomaterial can be co-planar with an adjacent outer surface of the pusher wire and/or implantable device. In one such exemplary configuration, the system includes recessed areas at the distal portion of the pusher wire and/or at the proximal portion of the implantable device for receiving the biomaterial.

Another illustrative configuration can comprise a biomaterial contact surface adapted to facilitate mating of the biomaterial with the implantable device and/or pusher wire. For example, the outer surface of the pusher wire can include a plurality of protrusions and/or depressions designed to increase the area to which the biomaterial can adhere.

The delivery system can further include structures to improve the stability of the system. For example, in one illustrative embodiment, a protective outer jacket encompasses a portion of the pusher wire and the implantable device. The jacket comprises a structure configured to receive a flushing agent. The biomaterial can at least partially contact the jacket, the pusher wire, and the implantable device. The protective jacket can be secured at one end to the implantable device or to the pusher wire. In an illustrative configuration, the protective jacket encompasses the length of the pusher wire.

In another illustrative embodiment, in addition to the biomaterial, a mechanical connection can mate the implantable device to the pusher wire. The mechanical connection can, for example, be a detachable mechanical interlock.

Also disclosed herein are methods for delivering an implantable device. The method can include providing an implantable device mated to a control wire via biomaterial, where the biomaterial inhibits decoupling of the implantable device from the pusher wire. A user can move the implantable device through a catheter by pushing the pusher wire. Delivering the implantable device can be achieved by pushing at least a portion of the implantable device and the pusher wire out of the distal end of the catheter and exposing the biomaterial to a flushing agent, thereby allowing the implantable device to decouple from the control wire.

Further disclosed herein are processes for securing an implantable device to a pusher wire. The process can include applying biomaterial to at least a portion of the implantable device and the pusher wire. In one embodiment, the location can be the distal end of the pusher wire and the proximal end of the implantable device. In another embodiment, the location can be a portion of the outer surfaces of the implantable device and the pusher wire.

Additional advantages will be set forth in part in the description which follows, and in part will be obvious from the description, or may be learned by practice of the description or claims that follow. The advantages will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate exemplary embodiments of the invention and, together with the description, serve to explain the principles of the invention.

FIG. 1A is a cut-away view of a catheter illustrating one embodiment of a implant delivery system described herein;

FIG. 1B is a cut-away view of a catheter illustrating another embodiment of a implant delivery system described herein;

FIG. 2 is a cross-sectional view of yet another embodiment of a implant delivery system described herein;

FIG. 3 is a cross-sectional view of another embodiment of an implant delivery system described herein;

FIG. 4 is a cross-sectional view of another embodiment of an implant delivery system described herein;

FIG. 5 is a cross-sectional view of another embodiment of an implant delivery system described herein;

FIG. 6 is a cross-sectional view of another embodiment of an implant delivery system described herein;

FIG. 7 is a cross-sectional view of another embodiment of an implant delivery system described herein;

FIG. 8 is a side view of yet another embodiment of an implant delivery system described herein;

FIG. 9A is a side view of an embodiment of an engaging member used with an implant delivery system described herein; and

FIG. 9B is a perspective view of the engaging member of FIG. 9A.

### DETAILED DESCRIPTION

Disclosed herein are methods and systems for delivering an implantable device to a target site, and more particularly, implantable device detachable from a pusher wire. Discussed below are a variety of delivery systems adapted to inhibit unwanted or premature detachment and/or jamming during delivery of the implantable device through a catheter. Wherever possible, the same reference numbers will be used throughout the drawings to refer to the same or like parts.

In one embodiment, a biomaterial can inhibit detachment of the implantable device from the pusher wire during delivery of the implantable through a catheter lumen. When the implantable device reaches a target location, the biomaterial can be removed or sufficiently weakened to allow delivery. For example, the biomaterial can be removed and/or weakened by absorbing, resorbing, dissolving, dispersing, separating, degrading, and/or digesting into the surrounding environment. Thus, the term biomaterial is used to indicate a biocompatible material with the capability of bioabsorbing, bioresorbing, dissolving, dispersing, separating, degrading, or digesting into a physiological environment. As described in more detail below, the biomaterial can include a variety of non-toxic or low-toxic materials.

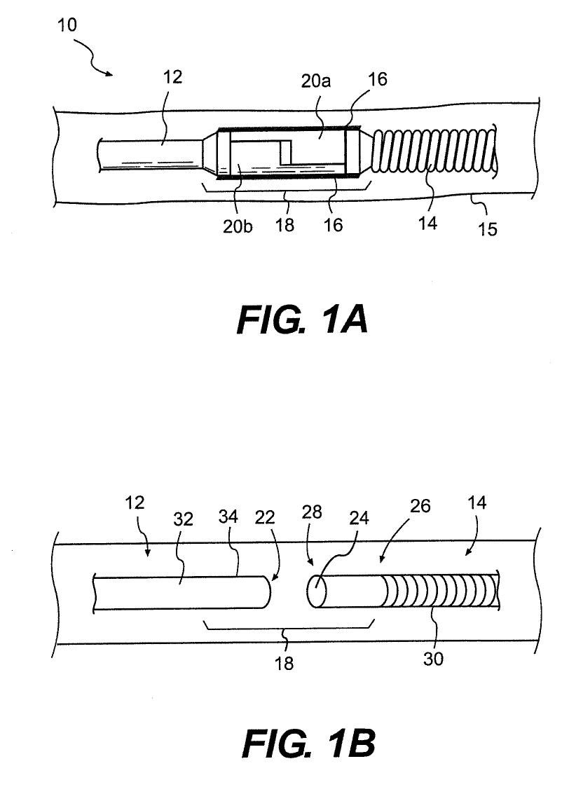

FIGS. 1A and 1B generally depict a system 10 for delivering an implantable device, in this instance, an embolic coil 14. While the following description refers to an embolic coil, it should be understood that a variety of implantable devices can be delivered with the system described herein. Coil 14 is delivered through a lumen of a medical device, in this case catheter 15, via a pusher wire 12, where the interface between coil 14 and pusher wire 12 is defined by a mating region 18. As shown in FIG. 1A, mating region 18 can include a mechanical interlock comprising first and second engaging members 20a, 20b. As described in more detail below, the mechanical interlock can work with a biomaterial 16 to inhibit detachment of the coil 14 from pusher wire 12 during delivery of the coil through catheter 15. Alternatively, as illustrated in FIG. 1B, system 10 does not include a mechanical interlock. Instead, mating region 18 is defined, at least in part, by contact surface 22 on pusher wire 12 and opposing contact surface 24 on coil 14.

Coil 14 can include a generally elongate body 26 extending from a proximal end 28, for interfacing with pusher wire 12, to a distal working portion 30 that includes a thrombosis inducing structure. Pusher wire 12 can include the variety of conventional pusher wires for moving implantable devices through a lumen of a medical device. In one aspect, pusher wire 12 includes an elongate body 32 that extends to a distal end 34. With respect to FIG. 1B, coil body 26 and pusher wire body 32 both have a generally cylindrical shape. However, one skilled in the art will appreciate that coil body 26 and pusher wire body 32 can have a variety of alternative shapes depending on the choice of catheter, the interface between coil 14 and pusher wire 12, and/or the intended use of system 10.

Contact surfaces 22, 24 allow a user to move coil 14 distally by applying pressure on pusher wire 12. In one embodiment, a biomaterial 16 mates the pusher wire and coil to allow a user to control additional degrees of freedom of movement of the coil. For example, the coil can be moved proximally, distally, and/or rotated by manipulating the pusher wire. In addition, or alternatively, the biomaterial 16 can inhibit jamming of the pusher wire and coil by keeping the contact surfaces 22, 24 aligned with one another.

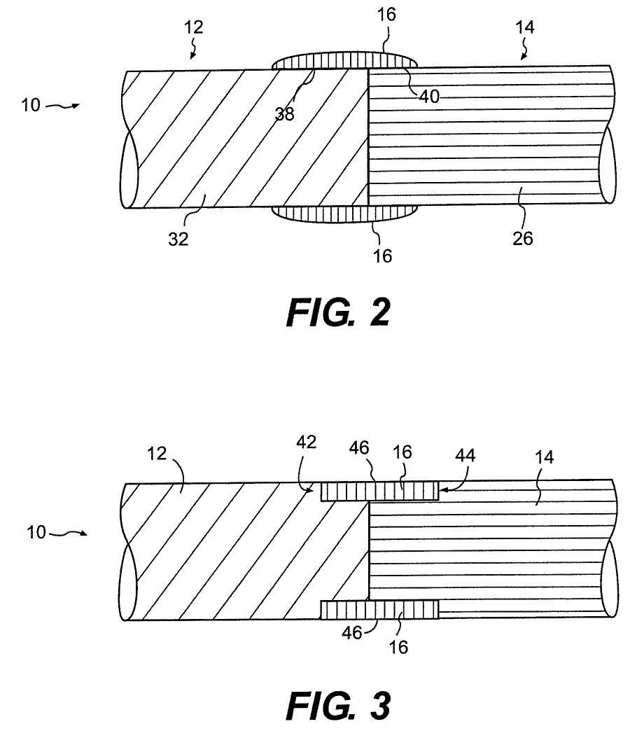

Referring now to FIG. 2, a biomaterial 16 can extend from pusher wire 12 to coil 14 to releasably connect the pusher wire and coil. As illustrated, biomaterial 16 can extend over at least a portion of an outer surface 38 of the pusher wire body 32 and over at least a portion of an outer surface 40 of coil body 26. In one aspect, biomaterial 16 adheres to and/or frictionally engages with outer surfaces 38, 40 to limit the movement of coil 14 relative to the pusher wire 12.

Biomaterial 16 can have a variety of configurations. In one aspect, biomaterial 16 extends around the full circumference of mating region 18. For example, the biomaterial 16 can have a generally tubular shape. Alternatively, only a portion of the interface between the pusher wire and coil is covered. In addition, the biomaterial 16 can be a continuous body or can be defined by several individual bodies (not illustrated) that extend between the pusher wire and coil.

As shown in FIG. 2, when the biomaterial 16 is positioned on the outer surface of the implantable device and/or pusher wire, the biomaterial 16 increases the diameter of the coil and/or pusher wire. In some cases it is desirable to minimize the outer diameter of catheter 15, which is limited by the need to provide sufficient inner lumen diameter for the passage of system 10. Accordingly, in another embodiment disclosed herein, the biomaterial 16 can be at least partially recessed in the outer surfaces 38, 40 of the implantable device and/or pusher wire to reduce the outer diameter of the biomaterial 16.

FIG. 3 depicts system 10 wherein the bodies of pusher wire 12 and coil 14 each contain an area of reduced diameter that defines recessed areas 42, 44, respectively. Depending on the depth of recessed areas 42, 44 and/or the choice of biomaterial 16, an outer surface 46 of biomaterial 16 can be flush or substantially flush with adjacent portions of the outer surfaces of pusher wire 12 and coil 14.

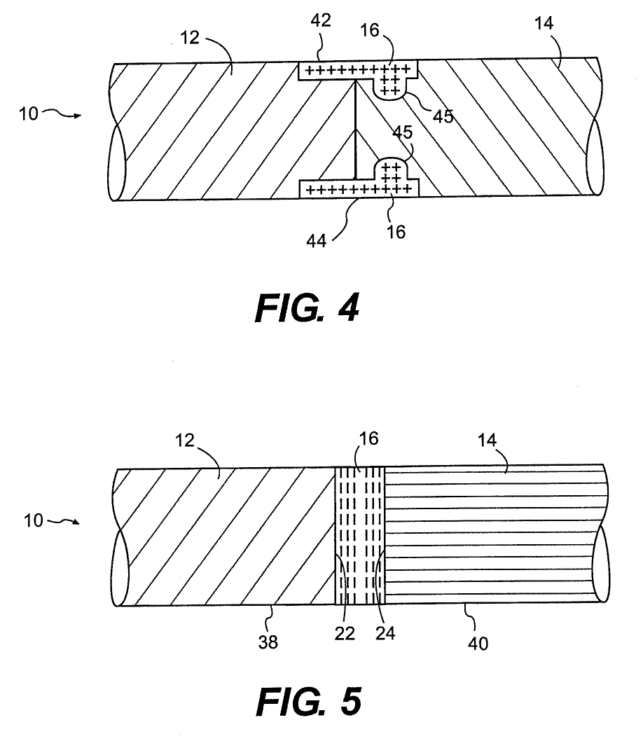

In another embodiment, the outer surfaces 38, 40 of the coil and/or pusher wire can includes surface features adapted to facilitate mating of the biomaterial 16 to the coil and/or pusher wire. FIG. 4 illustrates one embodiment where the recessed area includes a detent 45 in which a portion of the biomaterial 16 sits. In an alternative or additional aspect, outer surface 38 and/or 40 can include a protrusion that mechanically engages biomaterial 16. In yet another aspect, system 10 can include a texturized surface having multiple detents and/or protrusions to facilitate mating of the biomaterial 16 to the coil and/or pusher wire. Biomaterial 16 can, in one aspect, mechanically mate to at least one of the coil and pusher wire. For example, the recessed area can have a shape that traps a portion of the biomaterial 16.

In another embodiment disclosed herein, at least a portion of the biomaterial 16 can be positioned between contact surfaces 22, 24. FIG. 5 depicts biomaterial 16 mated to contact surfaces 22, 24 and extending between coil 14 and pusher wire 12. In one aspect, the outer surface of biomaterial 16 is flush with the outer surface of the pusher wire and the implantation device. In yet another embodiment, the biomaterial 16 can extend over a portion of outer surfaces 38, 40 and contact surfaces 22, 24. In addition, as discussed above with respect to outer surfaces 38, 40 of the pusher wire and coil, contact surfaces 22, 24 can include surface features to facilitate mating of the biomaterial 16 to the pusher wire and/or coil.

Positioning the biomaterial 16 as an intermediary between the pusher wire and the implantable device decreases the exposed surface area of the biomaterial 16. Thus, positioning the biomaterial 16 between the pusher wire and coil can slow the speed at which the biomaterial 16 degrades. In addition, such a position can protect the biomaterial 16 from damage.

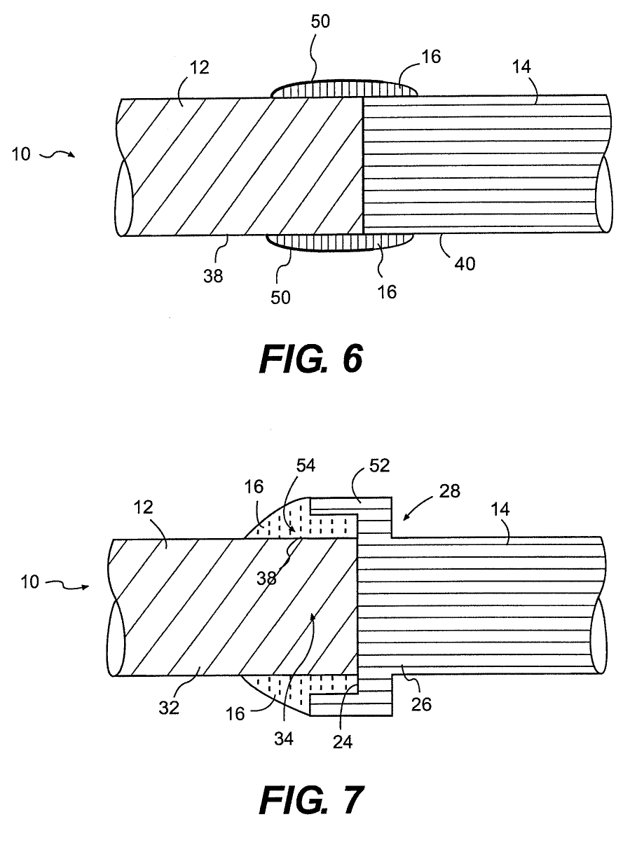

In a further embodiment, an outer jacket, sleeve, or sheath (these terms are used herein interchangeably) can protect the biomaterial 16. For example, the jacket can be designed to reduce or prevent damage to biomaterial 16 during manufacturing, shipping, and/or delivery of the implantable device through a medical device. FIG. 6 depicts implantable device delivery system 10 including jacket 50 extending over a portion of biomaterial 16. The protective jacket can be secured to the coil and/or to the pusher wire and extend over at least a portion of the mating region including biomaterial 16.

In one aspect, the jacket is removed prior to insertion of the implantable device into a catheter lumen. For example, jacket 50 can be releasably mated to the outer surfaces 38, 40 of pusher wire 12 and coil 14. In another aspect, jacket 50 remains attached to system 10 during delivery of implantable device. FIG. 6 illustrates jacket 50 attached to pusher wire 12, but not extending over the full length of biomaterial 16. In particular, a portion of biomaterial 16 remains exposed. During delivery through a catheter and/or at the target delivery location, the exposed portion of the biomaterial 16 allows for weakening and/or removal of the biomaterial 16. Once the implantable device is delivered, the jacket, which is attached to the pusher wire, is removed along with the pusher wire. While the illustrated embodiment shows a distal portion of 16 exposed, jacket 50 can have additional or alternative openings to expose biomaterial 16. For example, jacket 50 could include a series of apertures along its length (not illustrated).

The jacket can be constructed from a variety of materials depending on the choice of biomaterial 16, the location of the biomaterial 16, and/or the intended use of system 10. In one aspect, the jacket is formed of flexible or semi-rigid material. Alternatively, the jacket can be formed of a rigid material such as, for example, platinum or stainless steel.

As mentioned above, the mating area defined by pusher wire body 32 and coil body 26 can have a variety of configurations. FIG. 7 illustrates a cross-sectional view of coil and pusher wire bodies 26, 32 having a male/female mating configuration. The proximal end 28 of coil 14 includes contact surface 24 recessed within the proximal end of the coil body. A sidewall 52 surrounds contact surface 24 and defines a recessed area 54 into which the distal end 34 of pusher wire 12 can sit.

In one aspect, recessed area 54 has an inner diameter larger than the outer diameter of pusher wire body 32. Biomaterial 16 can be positioned in the area provided between the outer surface 38 of pusher wire body 32 and the inner surface of recessed area 54. This configuration of the mating area can provide additional surface area for mating biomaterial 16 with the coil and pusher wire body. While the pusher wire and coil are illustrated as having a male and female configuration, respectively, in another aspect, the configuration of the pusher wire and coil could be reversed.

In another aspect, the system can include an implantable device secured to a pusher wire by both biomaterial 16 and a detachable link. As mentioned above, the mating area 18 between the pusher wire and the coil can be defined by a detachable link comprising first and second engaging members. The engaging members are adapted to mechanically interlock, such that the pusher wire can move the coil proximally and distally. However, once the detachable link has exited the distal end of the catheter, the engaging member can self-detach.

While engaging members 20a, 20b (FIG. 1A) can inhibit relative longitudinal movement between the pusher wire and coil (and possibly some relative rotational movement), the engaging member do not prevent relative radial movement of the engaging members. As such, there is the possibility that the engaging members could detach prematurely and/or jam. The biomaterial 16 can inhibit unwanted detachment and/or jamming during delivery by limiting relative movement (particularly relative radial movement) between the engaging members.

In addition, the biomaterial 16 can hold the detachable link together for a short time period after the detachable link leaves the distal end of the catheter. The ability to control the detachable link beyond the distal end of the catheter allows a physician additional latitude in delivering the coil. For example, while the detachable link is held together by the biomaterial 16, a physician can reposition the detachable link and/or withdraw the detachable link back into the catheter.

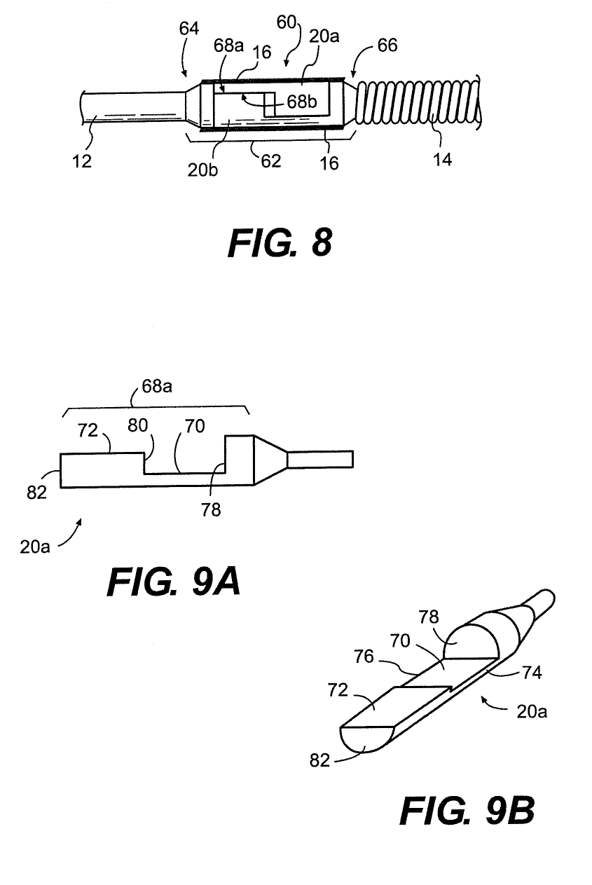

FIGS. 8 through 9B illustrate an exemplary embodiment of detachable link 60 generally including a body 62 formed from at least interlocking members 20a, 20b. Body 62 can have a generally elongate shape extending from a proximal portion 64 to a distal portion 66. In one aspect, proximal and distal portions 64, 66 of body 62 can be integrally formed with pusher wire 12 and coil 14, respectively. Alternatively, body 62 can be fixedly mated with the coil and pusher wire. For example, the coil and pusher wire can be welded, adhered, and/or mechanically mated with body 62.

Engaging members 20a, 20b, can have a variety shapes and/or sizes that provide a detachable connection that self detaches after exiting the distal end of a catheter. In one aspect, engaging members 20a, 20b can mechanically interlock with one another. For example, the engaging members can be configured as interlocking arms that are held together by biomaterial 16 and/or catheter 15.

In one embodiment, the engaging members are generally configured such that opposed mating surfaces 68a, 68b of the engaging members 20a, 20b reversibly accept a portion of the adjacent engaging member 20a, 20b. The mating surfaces 68a, 68b of the engaging members can be configured to transmit longitudinal forces (i.e., pushing/pull) so that a user can move coil 14 through catheter 15. FIGS. 9A and 9B illustrate an exemplary engaging member (e.g., engaging member 20a) having a mating surface 68a comprising a receiving area 70 and an extension portion 72. In the illustrated embodiment, receiving area 70 has open sides 74, 76 and is bounded by end surfaces 78, 80.

In one aspect, vertical surfaces (i.e., surfaces transverse to the longitudinal axis of the detachable link) of receiving area 70 and extension portion 72 allow coupled engaging members 20a, 20b to push/pull one another. For example, end surface 78 can provide a vertical contact area for pulling on a similar vertical surface on an adjacent engaging member (e.g., engaging member 20b). When engaging member 20a is pushed, end surface 80 can be pushed by a surface (e.g., surface 82) on an adjacent engaging member. While surfaces 78, 80, and 82 are illustrated as vertical, in another embodiment, at least one of the surfaces of engaging members can have a ramped configuration. In addition, while surfaces 78, 80, 82 are illustrated as planar, one or more of the surfaces could have a non-planar configuration such that receiving area 70 and extension portion 72 have a non-rectangular shape. One skilled in the art will appreciate that extension portion 72 and receiving area 70 can have a variety of shapes including, for example, a circular, oval, rectangular, multi-sided, or irregular shapes that are adapted to mate with receiving areas and protrusions of a corresponding, or different, shape.

In one embodiment, mating surfaces 68a, 68b allow pushing/pulling of coil through catheter, but do not prevent relative radial and/or rotational movement between engaging members 20a, 20b such that surfaces 68a, 68b can move away from one another and/or rotate relative to one another.

Biomaterial 16 can inhibit at least one degree of freedom of engaging member 20a relative to engaging member 20b. For example, biomaterial 16 can mate with the outer surfaces of engaging members 20a, 20b, preventing relative movement between the engaging members. In another aspect, biomaterial 16 can mate with the mating surfaces 68a, 68b of engaging members 20a, 20b to prevent relative movement. FIG. 8 illustrates biomaterial 16 extending over and between engaging members 20a, 20b. Regardless of the location of biomaterial 16, the biomaterial 16 can mechanically engage, frictionally engage, and/or adhere to engaging members 20a, 20b.

Detachable link 60 and biomaterial 16 can include the various features described above with respect to the different embodiments of the mating section between pusher wire 12 and coil 14. For example, the outer surface of one or both of engaging members 20a, 20b, can include a recess for receiving at least a portion of biomaterial 16.

A variety of biomaterials can be used with the systems and methods described herein. In one aspect, the biomaterial can be selected to maintain a minimum strength. This minimum strength may be greater than the force needed to deliver and retract the coil. For example, the strength of the biomaterial can be at least about 0.1 lb_{f}, at least about 0.3 lb_{f}, at least about 1 lb_{f}, or at least about 2 lb_{f} prior to delivery through a catheter lumen. The strength of the biomaterial can be weakened during the transit through the delivery catheter. For example, the presence of fluids within the catheter lumen can sufficiently weaken the biomaterial to allow delivery of the implantable device when it reaches a target location.

In another aspect, the biomaterial is flexible or semi-rigid to allow some limited movement between the pusher wire and coil. The delivery catheter can follow a tortuous path that results in a curved pathway. Allowing some movement between the coil and pusher wire facilitates moving the system around corners in the catheter lumen. The biomaterial, in one exemplary embodiment, can be sufficiently flexible to allow some limited bending at the interface between the pusher wire and the coil.

Generally, the biomaterial will begin to degrade after exposure to a flushing agent. The flushing agent can include biological materials present within a body and/or fluids delivered by a user. In particular, the flushing agent can comprise one or more fluids capable of initiating removal and/or weakening of the biomaterial. In some embodiments, the source of the flushing agent is external from the patient and can include an active component effecting removal of the biomaterial, for example, a liquid, a solid component suspended in a solvent, a solid component dissolved in a solvent, an emulsion, a gel, or any other fluid available to the skilled artisan. In another embodiment, when the biomaterial is aqueous-soluble or aqueous-dispersible, the flushing agent can be physiological in vivo fluid, such as blood. Thus, delivering the system outside the distal end of the delivery catheter at a delivery location in a human or animal exposes the biomaterial to the in vivo flushing agent.

The flushing agent can alternatively, or additionally, contact the biomaterial inside the catheter. For example, retrograde blood flow can result in the presence of biological fluids within the catheter lumen. The blood can start to remove and/or weaken the biomaterial as the biomaterial is delivered through the catheter. Similarly, saline solution can be delivered through the catheter to initiate removal of the biomaterial during the passage of the biomaterial through the catheter.

The rate at which the biomaterial degrades can be varied to depending on the intended use of system 10. In some instances, the decoupling takes place in about 10 minutes or less after exposure to the flushing agent, about 5 minutes or less, about 4 minutes or less, about 3 minutes or less, about 2 minutes or less, or about 1 minute or less. In other embodiments, the decoupling rate is about 5 seconds or less, about 4 seconds or less, about 3 seconds or less, about 2 seconds or less, or about 1 second or less.

To accomplish this decoupling action, the biomaterial can include polymers comprising hydrolytically instable linkages. The polymer may be modified using techniques known in the art to arrive at the desired degradation rate or range of degradation rates. In one embodiment, the polymer is a copolymer having a plurality of a first monomeric species and a plurality of a second monomeric species. The ratio of the first monomer, for example, lactide, to the second monomer, for example, glycolide, may be varied to obtain a copolymer with the desired degradation rate. In yet another embodiment, the polymer may be a graph copolymer, including a monomeric species attached to a polymer backbone. The ratio of that species to the backbone may be adjusted to arrived at a material with the desired properties. Exemplary polymer backbones may include, without limitation, poly(ethylene glycole), PVA, dextrane, or charged dextrane.

In yet another embodiment, exemplary polymers having hydrolytically instable linkages include, without limitation, poly(hydroxy acid), poly(lactide), poly(glycolide), poly(trimethylene carbonate), polycaprolactone, poly(dioxanone), and polydepsipeptides. The flushing agent can comprise components suitable to initiate dissolution of the biomaterial in the desired timeframe.

The biomaterial can comprise an amorphous layer or discrete components bound together with a binding agent. For example, suitable discrete components include microfibers, nanofibers, or nanoparticles. The binding agent can be selected to increase the rate of decoupling of the implantable device from the pusher wire.

In one embodiment, the biomaterial is aqueous-soluble or aqueous-dispersible. In this instance, human or animal blood is the aqueous environment that serves as the flushing agent to initiate dissolution or dispersement. The aqueous-soluble or dispersible material includes materials capable of dissolving or dispersing within about ten minutes when a cube of the material having edges measuring about 0.05 inches is exposed to human blood at normal body temperature. Exemplary materials include, for example, at least one of natural sugars, saccharides, starches, other carbohydrates, sugar alcohols, and polymers. Suitable sugar alcohols include, for example, at least one of mannitol, iditol, glucitol, rabitol, heptitol, octitol, arabinitol, betitol, bornesitiol, dambonitol, inositol, laminitol, onoitol, pinitol, and sorbitol. Other aqueous-soluble or dispersible materials can include non-cross-linked gelatin, poly(vinyl alcohol) polymers or copolymers, poly(vinylpyrrolidone) polymers or co-polymers, soluble acrylates, soluble ethers, and soluble polyesters. In another embodiment, the biomaterial can comprise a poly(lactic acid) polymer or co-polymer. In a further embodiment, the biomaterial can include additives selected to modify the rate of decoupling of the implantable device from the pusher wire.

The total thickness of the coating or coatings will vary depending on the chosen biomaterial and intended use of the system. In one exemplary embodiment, the coating(s) thickness ranges from about 0.0008 inches to about 0.045 inches, from about 0.001 inches to about 0.038 inches, and from about 0.01 inches to about 0.03 inches.

Biomaterial 16 can comprise more than a single layer, including two or more layers. Where more than one layer of biomaterial is present, each layer of biomaterial can be the same material or can be different materials having varying rates of removal upon exposure to the flushing agent. In addition, the different layers can be absorbed, dissolved, and/or degraded by different substances. The materials and layer thickness can be chosen to control the decoupling profile of the biomaterial 16. For example, an outer layer can be configured to degrade slowly via saline solution during insertion of the system through a catheter, while an inner layer can be configured to degrade rapidly once the outer layer is removed. In another aspect, a protective outer layer can prevent damage to an inner layer during delivery of the system to a user. The user can remove the outer layer prior to insertion of the implantable device (e.g., with saline solution). Once delivered to a target location within a patient, the inner layer can be degraded via a biological fluid.

Further provided herein is a method for delivering an implantable device. In one embodiment, the above described system is used to deliver an embolic coil to a target destination and then detach the coil. The embolic coil can first be moved from an introducer to a catheter by actuating the pusher wire. The biomaterial allows a user to control coil movement while inhibiting accidental detachment of the coil caused by variations in lumen diameter sometimes found at an interface between an introducer and a delivery catheter.

Once the coil is positioned in the catheter, the user can move the coil along the inner lumen of the catheter until the coil is proximate to the distal end of the catheter. At this point, the user can wish to determine the location of the coil within the catheter and/or relative to an anatomical feature. The delivery method can include the step of visualizing the relative location of the implantable device and the distal end of the catheter. For example, an imaging technique, such as x-ray, MRI, CT, PET, SPECT and combinations of any of the foregoing, can be used to visualize the coil.

In addition, or alternatively, system 10 can be adapted to provide the user with tactile feedback once the coil reaches a distal portion of the catheter. For example, a distal portion of a catheter lumen can include a surface feature that will cause increased resistance to movement of a portion of system 10 through a catheter. The surface feature can include, for example, a ribbed texture, a different material providing a different coefficient of friction, or a combination of both. When the increased resistance is felt, the user will be alerted to the location of the implantable device within a delivery catheter.

Once system 10 is positioned at the desired location, the user can detach and deposit the coil. In one embodiment, the user can actuate the pusher wire to expose the biomaterial to a flushing agent. With the biomaterial 16 exposed to body fluids, such as blood, the biomaterial 16 degrades and allows the coil to detach from the pusher wire. In one aspect, the user can move the pusher wire proximally and distally to speed up degradation of the biomaterial 16.

In an alternative, or additional aspect, the user can apply a flushing agent to the biomaterial 16 to initiate and/or speed dissolution of the biomaterial 16. In one embodiment, the flushing agent is supplied to the catheter containing system 10. In another embodiment, a second catheter is positioned in a location suitable to disperse flushing agent to the biomaterial. An inner lumen in the second catheter can transport the flushing agent to the coil delivery location. In an alternative embodiment, the delivery catheter utilized to delivery the implantable coil can include a second inner lumen. This second inner lumen can provide a source of flushing agent used to controllable decouple coil 14 from pusher wire 12.

Yet another aspect relates to processes for preparing the implantable device delivery systems disclosed herein. The process can comprise providing an implantable medical device and a pusher wire and applying biomaterial as disclosed herein to secure the implantable medical device and the pusher wire. In one embodiment, the biomaterial is at least one layer coating a portion of the outer surfaces of the implantable medical device and the pusher wire. In yet another embodiment, the biomaterial is located between the distal end of the pusher wire and the proximal end of the implantable medical device.

The step of applying the biomaterial can include techniques including, for example and without limitation, spraying, dip coating, spin coating, and extrusion.

It is intended that the specification and examples be considered as exemplary only, with a true scope and spirit of the invention being indicated by the following claims.

What is claimed is:

1. An implant delivery system comprising:
an implantable medical device including a body extending between a proximal and distal portion, the proximal portion having a first contact surface and the distal portion having a distal working section;
a pusher wire including an elongate body extending to a distal portion having a second contact surface; and
a biomaterial mated with the pusher wire and the implantable medical device, wherein the biomaterial is configured to decouple from the implantable medical device after exposure to a flushing agent.

2. The system of claim 1, wherein the first and second contact surfaces are adapted to mechanically engage with one another.

3. The system of claim 2, wherein the biomaterial mates with outer surfaces of the implantable medical device and the pusher wire.

4. The system of claim 1, wherein the biomaterial is positioned between the first and second contact surfaces.

5. The system of claim 1, further comprising an elongate medical device having an inner lumen.

6. The system of claim 1, further comprising an outer jacket.

7. The system of claim 6, wherein the jacket covers at least a portion of the pusher wire and at least a portion of the implantable device.

8. The system of claim 7, wherein the jacket is attached to the pusher wire.

9. The system of claim 1, wherein the biomaterial comprises a polymer and a hydrolytically instable linkage, wherein the linkage decouples after exposure to the flushing agent.

10. The system of claim 1, wherein the biomaterial is at least one of aqueous-soluble or aqueous-dispersible.

11. The system of claim 1, wherein the biomaterial includes at least two layers of material, wherein the at least two layers absorb at different rates.

12. The system of claim 1, wherein an outer surface of the pusher wire body includes a recessed area and at least a portion of the biomaterial is positioned within the recessed area.

13. The system of claim 12, wherein the recess extends around the full circumference of the pusher wire body.

14. The system of claim 1, wherein an outer surface of the implantable device body includes a recessed area and at least a portion of the biomaterial is positioned within the recessed area.

15. A implantable device linking system, wherein the system comprises:
a detachable link comprising an elongate body member including first and second interlocking members, the first interlocking member including a first contact surface having at least one protrusion, and the second interlocking member including second contact surface having at least one protrusion; and
biomaterial mated with the first and second interlocking members, wherein the biomaterial inhibits relative movement between the first and second interlocking members.

16. The system of claim 15, wherein the biomaterial mates with outer surfaces of the first and second interlocking members.

17. The system of claim 15, wherein the biomaterial is positioned between the first and second contact surfaces.

18. The system of claim 15, further comprising an elongate medical device having an inner lumen.

19. The system of claim 15, wherein the first member is mated with a pusher wire and the second interlocking member is mated with an embolic coil.

20. The system of claim 15, wherein the biomaterial comprises a polymer and a hydrolytically instable linkage, wherein the linkage decouples after exposure to the flushing agent.

21. The system of claim 15, wherein the biomaterial is at least one of aqueous-soluble or aqueous-dispersible.

22. The system of claim 15, wherein the biomaterial includes at least two layers of material, wherein the at least two layers absorb at different rates.

23. A method for delivering a detachable implant, said method comprising
providing an implantable medical device secured to a pusher wire by biomaterial;
actuating the pusher wire to move the implantable medical device through a catheter; and
exposing the biomaterial to a flushing agent to allow the implantable medical device to detach from the pusher wire.

24. The method according to claim 23, wherein the step of exposing comprises at least one of:
delivering the medical device through a distal opening in the catheter; or
allowing an in vivo substance to contact the biomaterial; or
delivering saline solution through the catheter.
